(19) United States
(12) Patent Application Publication
(10) Pub. No.: US 2015/0355072 A1
(43) Pub. Date: Dec. 10, 2015

(54) Method for Enriching and Isolating Cells Having Concentrations Over Several Logarithmic Steps

(71) Applicant: SIEMENS AKTIENGESELLSCHAFT, München (DE)

(72) Inventors: Oliver Hayden, Herzogenaurach (DE); Michael Johannes Helou, Regensburg (DE); Lukas Richter, Hirschaid (DE)

(21) Appl. No.: 14/762,700

(22) PCT Filed: Jan. 15, 2014
(86) PCT No.: PCT/EP2014/050642

§ 371 Date: Jul. 22, 2015

(30) Foreign Application Priority Data
Jan. 22, 2013 (DE) 102013200927.5

(51) Int. Cl. G01N 15/14; G01N 33/543; G01N 33/58

(52) U.S. Cl. CPC G01N 15/1404; G01N 33/585; G01N 33/54326; G01N 2015/1006

(57) ABSTRACT

For flow cytometric measurement, a channel includes a magnetic sensor and is disposed downstream of a chamber. The chamber and the channel form a closed system, wherein an axis of the channel extends along a flow direction of the channel. A closed system is present if the chamber merges directly into the channel. A magnet, more particularly a permanent magnet, and a deflection device, are both arranged at a predefined side of the channel. Here, the deflection device has at least one segment. Each segment is arranged in a concentration region of the channel. Each segment has a guide for guiding cells toward the axis. The deflection device thus enables an enrichment of magnetically marked cells, which are pulled to the channel side by the magnet in the respective concentration region, on the axis and guidance of these cells along the axis to the magnetic sensor.

### RELATED CASES

The present patent document is a §371 nationalization of PCT Application Serial Number PCT/EP2014/050642, filed Jan. 15, 2014, designating the United States, which is hereby incorporated by reference. This patent document also claims the benefit of DE 10201320927.5, filed Jan. 22, 2013, which is also hereby incorporated by reference.

### FIELD

The present embodiments relate to magnetic flow cytometry.

### BACKGROUND

In currently known methods for flow cytometry, cells are focused in the center of the main channel by additional liquid flows in a channel and are thus isolated (sheath-flow principle). This isolation renders it possible to count the cells in a subsequent measurement step (e.g. by optical or impedimetric). By magnetically marking the particles to be quantified, it is possible to isolate and enrich the particles from a complex sample prior to the actual quantification.

However, in order to maintain the functionality of this technology, a significant amount of additional liquid that generates the sheath flow is required. This method is also connected with further preparations with, for example diluting steps and centrifuging steps, which are also connected with a risk of contamination. The diluting steps are necessary to match a count rate to the minimum and maximum count rate of a magnetic sensor (i.e. of the counter) of the cytometer.

### SUMMARY AND DESCRIPTION

The object to be achieved lies in the provision of a device and a method, by which a cytometric measurement, such as a determination of the concentration of cell samples, may be performed more quickly and with a lower risk of contamination.

This object is achieved by the device according to one or more embodiments for a flow cytometric measurement. Furthermore, the object is achieved by the method.

The present embodiment is based on the concept of performing a plurality of enrichment acts within a closed system. This is rendered possible by the structural design of a channel of the device and by the use of a deflection device in the channel. The embodiments enables targeted enrichment and isolation of cells with concentrations over several powers of 10 (from e.g. 10 to 10 000 cells/microliter). In particular, the one embodiment renders it possible to be able to measure possible cell concentrations over a plurality of powers of 10 in a single measurement process. Even in the case of a restricted dynamic response of the counter, this allows a large dynamic range to be covered in a single microfluidic channel within a complex sample, without the necessity of preceding diluting, isolation or enrichment acts.

To this end, the device according to one embodiment for a flow cytometric measurement includes a chamber and the channel, wherein the channel includes a magnetic sensor and is disposed downstream of the chamber. The chamber and the channel form a closed system, wherein an axis of the channel extends along a flow direction of the channel. A closed system is present if the chamber merges directly into the channel. The device is characterized by a magnet, more particularly a permanent magnet, and a deflection device, which are both arranged at a predefined side of the channel. Here, the deflection device has at least one segment. Each segment is arranged in a concentration region of the channel. Each segment has a guide for guiding cells toward the axis. The deflection device thus enables an enrichment of magnetically marked cells, which are pulled to the channel side by the magnet in the respective concentration region, on the axis and guidance of these cells along the axis to the magnetic sensor.

The object addressed above is likewise achieved by the method for enriching cells of a cell type to be detected in a cell sample for flow cytometry. Here, initially, magnetically marked cells of the cell type to be detected are provided. The marked cells are enriched in the channel of an embodiment of the device. To this end, the marked cells are pulled onto the predetermined side of the channel by the magnet in the channel. In the case of the laminar flow in the channel, at least some of the cells there are guided by the deflection device to the axis that extends along the flow direction of the channel and, as a result of this, are enriched on the axis. This enables an efficient enrichment of cells. Thus, even complex samples (i.e., non-purified samples), which contain a multiplicity of different cells and other particles, such as proteins, may be used for flow cytometry without intermediate acts, in particular in an undiluted manner.

The provision of the magnetically marked cells may include the marking of the cells. Here, two marking variants may be used.

In a first variant, the cells to be detected may be marked in an incubation act by mixing and/or stirring markers, in particular at least one antibody specific to the cell type to be detected. The antibody is connected to at least one magnetic marker and the cell sample in the chamber of the device. This variant is preferably used in the case of small overall cell concentrations of up to 10⁴ cells/microliter. A magnet arranged on the side of the chamber may assist the mixing and/or stirring process in this case.

In one embodiment of the device, a magnet is correspondingly arranged at a predetermined side of the chamber, which magnet assists the mixing and/or stirring process. In the case of cell samples with a larger overall cell concentration (e.g., of up to 10⁶ cells/microliter), it is alternatively possible for the at least one antibody specific to the cell type to be detected during the marking, which antibody is connected with at least one magnetic marker, to be provided in the channel. When a magnetic field is generated in the channel using a magnet arranged on the side of the channel, the antibody is enriched at the side of the channel. Thereupon, the cell sample is introduced into the channel and thus the at least one specific antibody is brought into contact with that portion of the cell sample that flows past the side of the channel in a laminar manner. As a result, there is partial marking of the cells contained in this portion, without dilution or purification acts being necessary. Consequently, a layer-by-layer marking of the desired cells is achieved in a laminar flow system. Alternatively, such a partial marking may also be performed in the chamber with the aid of the magnet arranged there.

As a result, only one layer (i.e., a defined fraction of the cell sample), such as e.g. 1% of the desired cells, is magnetically marked in the closed system in the case of a fluidic flow.

The deflection device may have one or more guide elements, which for example form a groove that directs the marked cells to the axis, for guiding cells in a segment. In a particularly advantageous embodiment of the device, at least one segment of the deflection device includes guide elements arranged at an angle to the axis for guiding cells. Together or on their own, said guide elements form at least one funnel shape tapering in the flow direction. This enables the deflection of magnetically marked cells to the axis of the channel and hence enables enrichment. By way of example, if it has wholly or partly of a ferromagnetic material, at least one guide element may form a V-shape tapering in the flow direction in a further embodiment of the device, promoting a magnetophoretic guide of magnetically marked cells.

Additionally or alternatively, at least two of the guide elements may be configured as walls for the cells and may be arranged offset along the axis in the flow direction and thereby form a mechanical guide toward the axis by virtue of forming boundaries for the movement path of the cells.

Additionally, a magnetic web may be arranged along the axis of the channel, in particular between two guide elements at the same axis level. As a result, the guidance of the magnetically marked cells on the axis of the channel is promoted.

Preferably, at least two concentration regions of the channel have different configurations. A cell sample may thus be enriched in a plurality of logarithmic acts. What this may achieve is that an enrichment is present that may be measured by a counter with a restricted dynamic response.

By way of example, the at least two concentration regions may differ in terms of predetermined height of the channel between the predetermined side and the side opposite to the predetermined side. As a result, a defined volume is determined in each concentration region. Different volumes in different concentration regions bring about a first enrichment of the cells by the magnet of the channel prior to the enrichment in the deflection element such that a statistically meaningful and a defined cell number is adjustable in each enrichment act at a given concentration. By contrast, the channel width is preferably constant for all concentration regions. In the case of a constant channel width, the at least two concentration regions may additionally or alternatively differ by a predetermined width of the deflection device (as measured perpendicular to the axis) and/or by a length of the respective segment of the deflection device along the axis of the channel. These dimensions determine the catchment area of the deflection device (i.e., influence the degree of enrichment).

In one embodiment of the method, a cytometry, in particular cell counting, is performed on the axis downstream of the deflection device by the magnetic sensor. Using the magnet sensor, it is possible, for example, to determine the concentration of the cell sample.

Determining the concentration of the cell sample by cytometry downstream of the deflection device in this case preferably includes counting the cells, which are enriched, from at least one of the concentration regions and flowing past on the axis, by the magnetic sensor. The concentration of the cell sample is established for at least one of the concentration regions from the counted value of the concentration region established when counting the enriched cells, the volume of the concentration region and the width of the segment of the deflection device in the concentration region. This is an efficient and time-saving measurement, the evaluation of which is quickly available. Naturally, selection is made here of at least the concentration region for which a counter value emerges that is large enough for a statistically relevant statement and smaller than the maximum count rate reliably registrable by the counter.

It may be necessary to establish the concentration region to which the cells currently counted at a given time belong. In a further embodiment of the method, the respective concentration region, for which a counter value is established, is established on the basis of an established time of the counting process of the magnetic sensor. It is thus possible to bring about an assignment of time intervals to the concentration region in a calibrated device. The flow speed should be taken into account in certain circumstances.

### BRIEF DESCRIPTION OF THE DRAWINGS

Below, the invention will once again be explained in more detail by way of specific exemplary embodiments on the basis of the attached drawings. In this respect:

FIG. 1 shows a sketch of an embodiment of a device in a cross section,

FIG. 2 shows sketches of a channel of a device according to one embodiment, wherein FIG. 1A shows a schematic cross section and FIG. 1B shows a schematic top view of the channel,

FIG. 3 shows sketches of various embodiments of deflection devices, wherein FIG. 3A shows a perspective illustration of a deflection device with a mechanical guide, and FIG. 3B and FIG. 3C show schematic top views of in each case a deflection device with a magnetophoretic guide,

FIG. 4 shows a sketch of a cell sample in the channel immediately after introducing the cell sample (FIG. 4A) and after applying a magnetic field (FIG. 4B) in an embodiment of a method,

FIG. 5 shows a sketch that elucidates the enrichment of the cells in a channel in an embodiment of a method, wherein FIG. 5A shows a top view of the channel at one point in time and FIG. 5B shows a top view of a segment of the deflection means at different times, and

FIG. 6 shows a sketch, in which the determination of a cell concentration in an embodiment of a method and four sections of the channel at different times are shown.

### DETAILED DESCRIPTION OF THE EMBODIMENTS

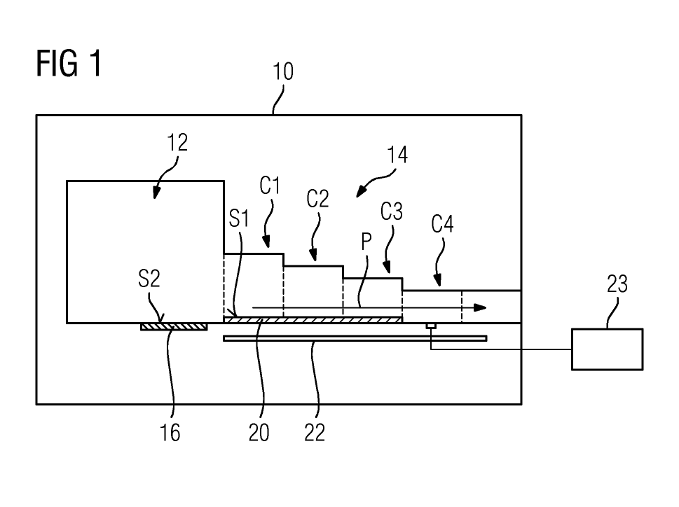

The exemplary embodiments explained in more detail below represent preferred embodiments. In the figures, functionally equivalent elements have the same reference sign. The coordinate systems K and K′ in the figures aid the orientation, wherein a vertical axis “z” and a horizontal axis “x” perpendicular thereto simplify the orientation in the cross section (FIG. 1A), and wherein the horizontal axis “x” and an axis “y” perpendicular to the horizontal axis “x” and to the vertical axis “z” simplify the orientation in the top view of the exemplary channel 14 (FIG. 1B). Here, the flow direction P points in the x-direction.

FIG. 1 shows a schematic diagram of an embodiment of a device 10 for performing a flow cytometric measurement. The device 10 includes a chamber 12 and a channel 14. The flow direction of the laminar flow present during cytometry is denoted by the arrow P, both in FIG. 1 and in the subsequent figures. On one side (S2), the chamber 12 may include a magnet 16, which for example is attached outside of the chamber 12 in this case. In accordance with the flow direction P, the channel 14 is disposed downstream of the chamber 12. The chamber 12 and the channel 14 form a closed system. The channel 14 furthermore includes a deflection device 20 and a magnet 22, in particular a permanent magnet, at a common channel side (S1) (e.g., the channel lower side).

A sensor 18 or a sensor array for cytometric measurement, in particular including a sensor chip, is attached to the channel 14 downstream of the deflection device 20 and it is configured to count magnetically marked cells. In particular, magnetoresistive resistors may be used in the sensor 18, preferably a GMR sensor. The sensor 18 and/or the sensor array is connected to an electronic evaluation device 23, which may be a processor of a computer for evaluating the measurement results.

Using the device it is possible, for example, to examine undiluted blood samples in respect of a concentration of, for example, the white blood cells.

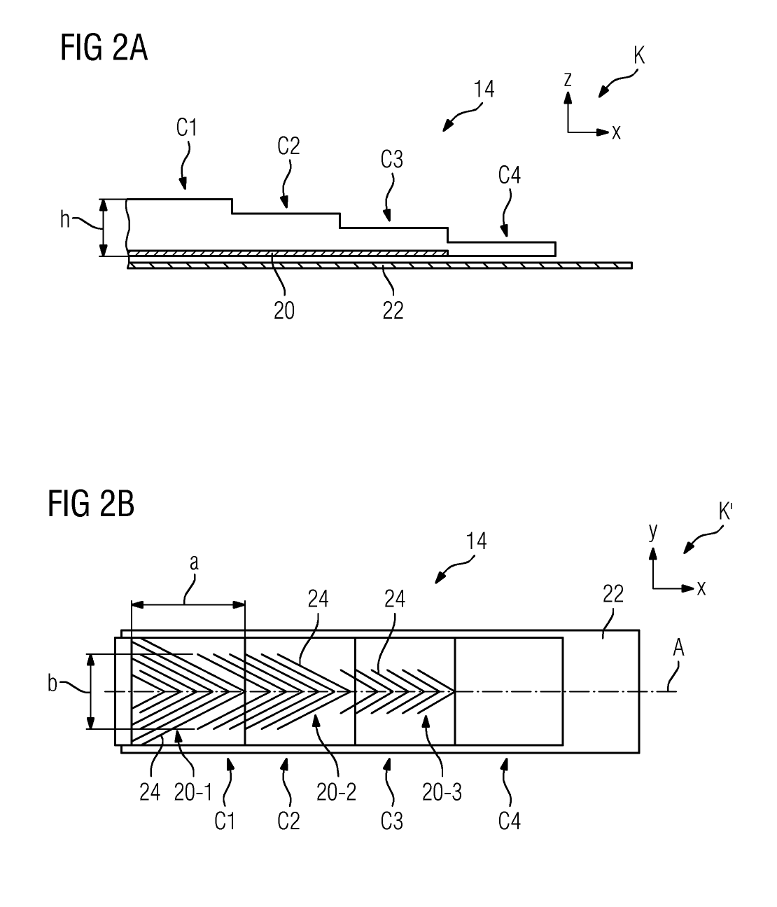

On the side of the channel 14 on which an enrichment of magnetically marked cells is desired, the channel 14 includes the deflection device 20, which in this case is arranged, for example, on the base of the channel 14. Arranged below the deflection device 20 is the magnet 22. By way of example, the channel 14 is divided into four concentration regions C1, C2, C3, C4. However, the number of concentration regions totaling four here is not mandatory (i.e. the channel 14 may be equipped with any number of concentration regions). The height h of the channel may differ in the various concentration regions C1, C2, C3, C4 of the channel 14. In the example shown in FIG. 2A, the height of the channel is highest, for example, in the concentration region C1, e.g. 100 μm, and reduces in each subsequent downstream concentration region C2, C3, C4. In the case of partial marking of the cells (see below), the height h is preferably stepwise equal in all concentration regions C1, C2, C3, C4.

FIG. 2B elucidates the design of the deflection device 20. In the exemplary channel 14, the deflection device 20 is subdivided into, for example, three segments 20-1, 20-2, 20-3, wherein each segment lies in a different one of the concentration regions C1, C2, C3. Here, each segment 20-1, 20-2, 20-3 may include a plurality of guide elements 24 arranged at an angle to the axis for guiding cells. For the sake of clarity, only some of the guide elements 24 are provided with the reference signs in FIG. 2B. The individual segments 20-1, 20-2, 20-3 each have a width b and a length a, which may respectively differ in segments 20-1, 20-2, 20-3 of different concentration regions C1, C2, C3. Preferably, the width b in this case decreases downstream such that wide segments are arranged upstream and narrow segments are arranged downstream. The width b of the deflection device 20 is that path that extends perpendicular to an axis A of the channel (dashed line) extending along the flow direction P of the channel 14. Thus, the width b is the catchment region of the deflection device 20 within the channel 14 along the horizontal “y”-axis. Accordingly, the length a is the length of the respective segment of the deflection device 20 along the axis A of the channel. The channel width, such as 100 μm, is constant in each concentration region C1, C2, C3, C4 in this case. By way of example, a segment 20-3 10 μm wide then enriches the marked cells 26 of 1/10 of the channel width (i.e., one logarithmic step less than for example the segment 20-1, if the latter comprises a width b of 100 μm).

The guide elements 24 may be arranged at an angle to the axis A, preferably at an acute angle between 0° and 90° in relation to the axis A, in particular between 0° and 45°, and, alone or together, form at least one funnel shape tapering in the flow direction P. By way of example, such a guide element 24 includes a wall that in the example here is attached to the inner side of the outer wall of the channel 14 and protrudes into the channel 14.

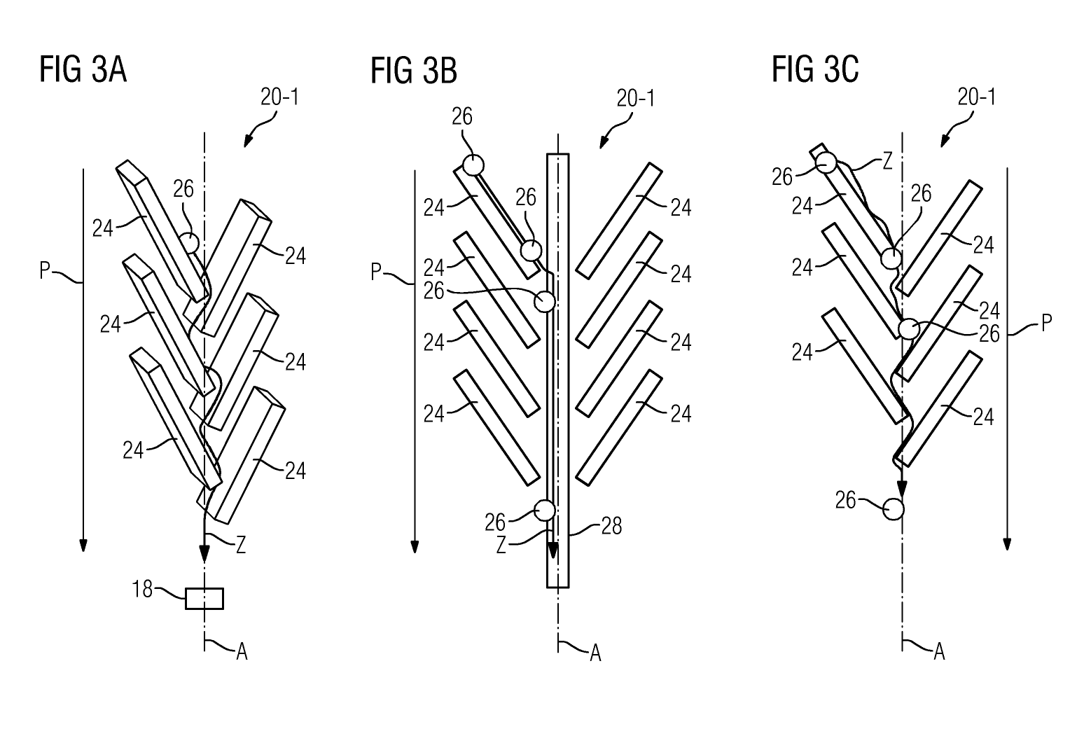

The guide elements 24 may be, for example, barriers made of, for example, photoresist, that mechanically guide the marked cells 26 and thus enrich these on the axis A, or, for example, ferromagnetic “fishbone structures” that magnetically focus and enrich the marked cells. A combination of both structures in one, or in different ones, of the segments 20-1, 20-2, 20-3 of the deflection device 20 is also possible. FIG. 2B shows the principle of the guide elements 24, which are depicted in FIG. 3 in a magnified manner. A guide element 24 guiding in a magnetophoretic manner is preferably made wholly or partly of a ferromagnetic material and/or has a wall height of, for example, 10 nm to 100 nm. Preferably, the wall height is less than 20%, in particular less than 10% of the mean cell diameter of the cells to be detected. The wall height of a guide element 24 for mechanical guidance is preferably greater than 20%, in particular 10%, of the diameter of the marked cell 26. Here, using the example of thrombocytes, this is based on a mean cell diameter of 3 μm.

An example for guide elements 24, for example, of a segment 20-1 of the deflection device 20, which form a mechanical guide, is shown in FIG. 3A. In this example, a number of the guide elements 24 are offset on the axis A in the flow direction P. The sensor 18 is also shown in FIG. 3A downstream of the deflection device 20. What is furthermore shown is a magnetically marked cell 26 and the movement of the marked cell 26, which is directed by the guide elements 24, by way of the arrow Z.

The figures denoted FIG. 3B and FIG. 3C each elucidate a further embodiment of the deflection device 20, in which, for example, a segment 20-1 is formed from guide elements 24 that bring about a magnetophoretic guidance of the marked cells 26. The guide elements 24 are wholly or partly formed from a magnetic material (e.g., strips of nickel). The wall height of such a guide element 24 is preferably 100 nm. The guide elements 24 are arranged at an angle to the axis A.

Two guide elements 24 of the exemplary embodiment shown in FIG. 3B, which are opposite one another in relation to the axis A, are not offset in relation to one another in the exemplary embodiment of FIG. 3B. A magnetic web 28, a so-called core, which may be wholly or partly formed from a magnetic material (e.g., nickel) extends there between I (i.e., on the axis A). Here, the magnetic material is preferably not exposed but covered by a passivation layer with a thickness of, for example, 100 nm and thus separated from the cell sample 30. The distance of the web 28 from those ends of the guide elements 24 that point to the axis A is preferably smaller than the diameter of the marked cell 26.

The guide elements 24 that are shown in the exemplary embodiment of FIG. 3C and are adjacent in relation to the axis A are offset in the flow direction P.

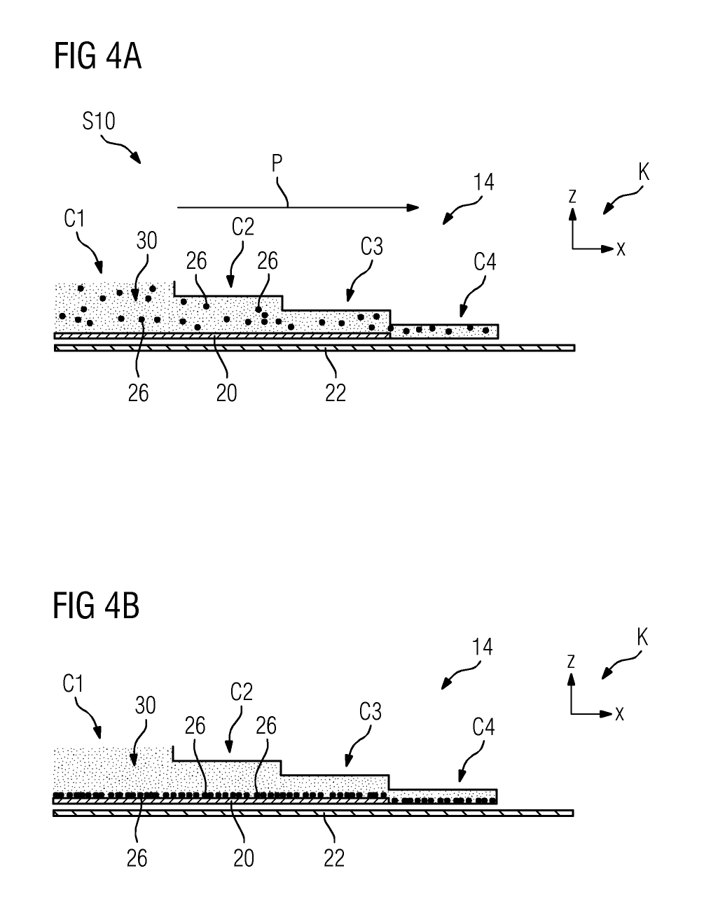

FIG. 4A to FIG. 6 elucidate movements of magnetically marked cells 26 in exemplary embodiments of the method (e.g., in flow cytometry), with the aid of a device 10. In these examples, for example, the coagulability of a whole blood sample is established with the aid of the method. To this end, the coagulation situation is estimated in the case of a patient, for example, prior to an operation, for example on the basis of the thrombocyte number in the whole blood sample, in order to determine hemostasis disorders.

In order to quantify specific cells, such as thrombocytes or CD4+ cells, within a cell sample 30, in particular within a complex sample such as a whole blood sample, said cells are firstly magnetically marked with a cell-specific marking since a complex sample contains different cells and particles (e.g., proteins). Marking is brought about by, for example, superparamagnetic markers, such as using magnetic particles (so-called microbeads), which are bound to cell-specific or particle-specific antibodies. The magnetic particles may have a diameter of less than 500 nm, preferably of less than 300 nm or of between 40 and 300 nm. For binding such markers to antibodies and for the production of cell-specific or particle-specific antibodies, a person skilled in the art may make use of conventional technologies. It is possible to perform a cell-specific marking within a complex sample with an overall cell concentration of, for example, 1 to 10⁶ cells/microliter with the aid of the device 10. In order to mark all cells of the desired cell type, the cell sample 30 and the marked antibodies are placed into the chamber 12 of the device 10. The antibodies then bind specifically to the cells to be enriched. This process, in which all cells to be enriched are marked, may be assisted by stirring the cell solution. Here, the magnet 16 may assist the stirring and/or mixing process. This variant is advantageous for cell samples with an overall cell concentration of up to 10⁴ cells/microliter.

Alternatively, the marking can also be partial, for example it is possible that only a fraction equaling 1% of all cells to be enriched are marked. This is advantageous, particularly in the case of a cell sample with a very high overall cell concentration, e.g. 10⁶ cells/microliter, because cell counting may otherwise overburden the dynamic range of the sensor 18. Here, the antibodies connected with the magnetic marker are put into the channel 14 of the device 10 first. A magnetic field may be generated by the magnet 22 at the predetermined side S1 of the channel 14 (see FIG. 1), the magnetic field of which magnet pulls the magnetically marked antibodies to the predetermined side S1 of the channel 14 and enriches them there. The magnet 16 is preferably attached to the outer side of the channel 14 such that it is not contaminated by the channel content. The cell sample 30 is subsequently introduced into the channel 14. The cells to be enriched (i.e., the thrombocytes in this example), which are situated in that portion of the cell sample 30 that flows past the predetermined side S1 of the channel 14 as a result of the laminar flow, thus come into contact with the marked antibodies. In this case, it is advantageous if there is a large number of markers per cell. All other cells do not come into contact with the marking. Depending on, for example, the form and size of the channel 14, it is thus possible to magnetically mark a defined fraction of cells, such as 1% or 10% of the thrombocytes, since these cells are available in a uniform spatial distribution in the channel 14 after the cell sample 30 is inserted. The marked cells 26 are available after the marking.

Whether mixing should be carried out or whether only a fraction should be marked may be estimated prior to carrying out the method using a blood count.

In the method act of enriching the marked cells 26, the cell sample 30, which contains magnetically marked cells 26, is guided by the laminar flow into the channel 14, for example, with the aid of a suction device and thus provided for enrichment, as shown in FIG. 4A (method act S10; in order to maintain clarity, only individual marked cells 26 are denoted by reference signs in FIGS. 4A to 6).

The degree of enrichment (or focusing) in each concentration region C1, C2, C3, C4 of the channel 14 is determined by the volume of the concentration region C1, C2, C3, C4 (i.e., by the height h of the channel 14, by the width b and/or the length a of the segment 20-1, 20-2, 20-3 of the deflection device 20 in a concentration region C1, C2, C3).

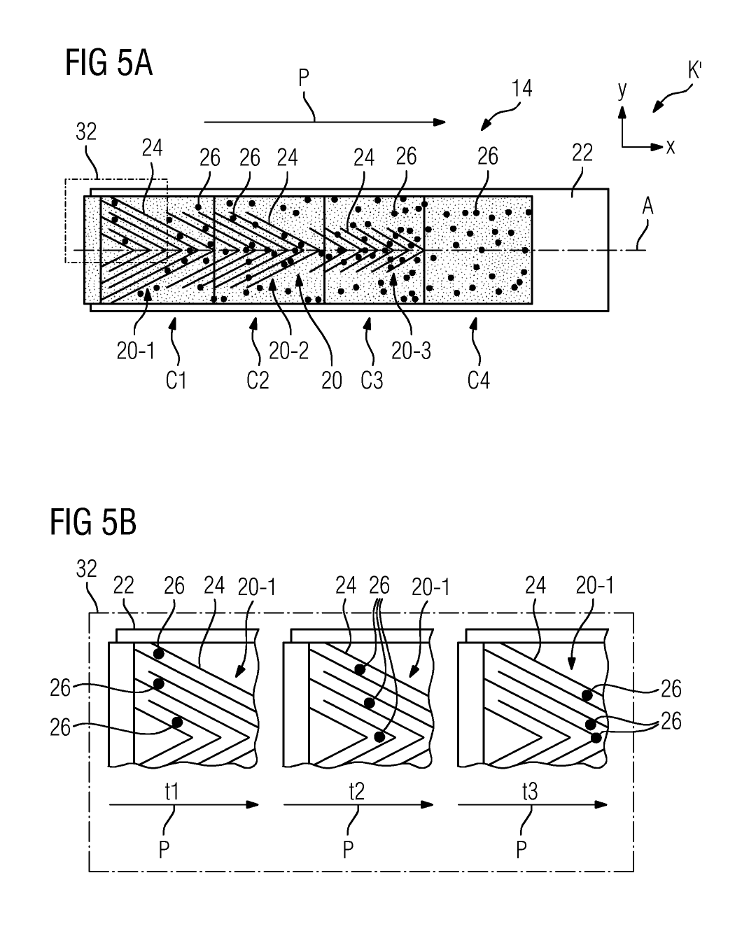

FIG. 4A shows, in an exemplary manner, a stochastic distribution of the marked cells 26 over the deflection device 20 immediately after introducing the cell sample 30 into the channel 14. When a magnetic field is generated by the magnet 22, the marked cells in each concentration region C1, C2, C3, C4 are pulled along the z-axis in the direction of the magnet 22 (FIG. 4B and FIG. 5A). The number of marked cells 26 that are pulled onto the respective segment 20-1, 20-2, 20-3, depends on the respective volume of the concentration region C1 (e.g., 140 to 200 microliters), C2 (e.g., 70 to 120 microliters), C3 (e.g., 30 to 60 microliters), C4 (e.g., 5 to 15 microliters) (i.e., on the respective height h of the channel 14). As a result, it is possible to set a defined and statistically meaningful number of marked cells 26 per concentration region C1, C2, C3, C4.

The sample 30 now flows through the channel 14 in the flow direction P. As a result of the laminar flow, the marked cells 26 on the deflection device 20 are pulled to the sensor 18.

After enrichment, the funnel shape of the deflection device 20 directs the marked cells 26 through a mechanical and/or a magnetophoric guide onto the axis A. This is elucidated in an exemplary manner in FIG. 5B, which shows the magnified section 32 of FIG. 5A at different successive times t1, t2 and t3. As a result, the marked cells 26 are enriched on the axis A. The width b determines the enrichment factor of the marked cells 26 of the cell sample 30 in the respective concentration region C1, C2, C3, C4.

V-shaped magnetophorically guiding guide elements 24, as were already described in relation to FIG. 2B, move the marked cells 26 along the axis A over the tapering ends of the V-shaped guide elements 24 due to the laminar liquid flow and, in this case, said marked cells adhere at the deflection device 20 due to the ferromagnetic properties of these guide elements 24.

Alternatively or additionally, a guide element 24, as shown in FIG. 3B and FIG. 3C, may guide the marked cell 26 in a magnetophoretic manner (see above). As a result of the gap between two guide elements 24, the cell 26 then glides in the direction of the sensor 18 along the flow direction P. As soon as the cell 26 is close to the axis, the cell 26 in FIG. 3B is attracted by the web 28. The flow then continues to press the cell A on the web 28 in the direction of the sensor 18 along the flow direction. Alternatively or additionally, a guide element 24 with a mechanical guide, such as a guide element as described in relation to FIG. 3A, forms a barrier by which the marked cell 26 is guided to the respectively next guide element 24 and finally to the axis A. As a result of the flow in the channel 14, the cell 26 is then moved on the axis A in the flow direction P (FIG. 3A, arrow Z).

The focused marked cells 26 are then directed directly to the sensor 18 on the axis A. Small magnetic strips, which may be attached in front of the sensor, can additionally align the cells 26 onto the sensor 18 and bind excess magnetic particles. As a result, enriched marked cells 26 are not deflected by the sensor 18 and the background noise during the measurement as a result of free markers is reduced.

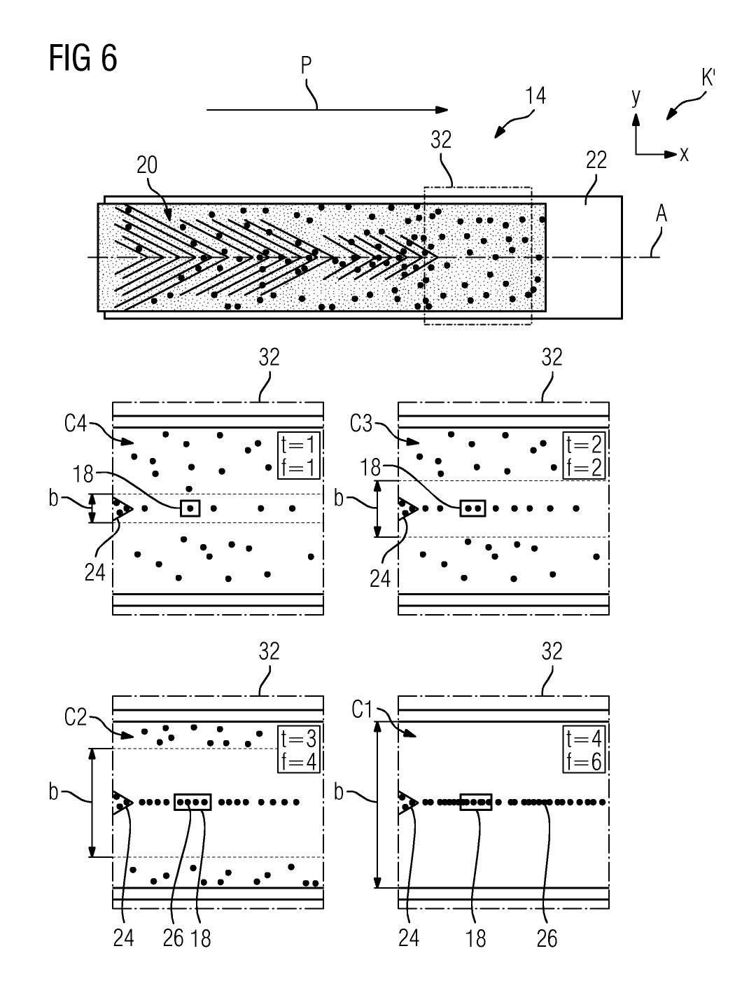

FIG. 6 shows a channel 14 of the device 10 in a top view, for example during a determination of the concentration. In the example, the thrombocyte number of the cell sample 30 is measured continuously. The section 32 is additionally shown at four different successive times t1 (“t=1”), t2 (“t=2”), t3 (“t=3”) and t4 (“t=4”) in a magnified manner. What can be seen in the magnifications of section 32 is the last guide element 24 of the segment 20-3, which has a width b of, for example, 10 μm. The marked cells 26, which were enriched in the respective concentration regions C1, C2, C3 and C4, flow over the sensor 18 on the axis A. At the time t1, marked cells 26 from the concentration region C4 are directed over the sensor 18. In this example, the concentration region C4 does not include a segment of the deflection device 20. Thus, only those cells 26 that are randomly situated on the axis A are registered by the sensor 18.

At a subsequent time t2 (“t=2”), the enriched marked cells 26 from the concentration region C3 are directed over the sensor 18, and said cells are counted by the evaluation means 23 by said sensor.

The wider and/or longer a segment 20-1, 20-2, 20-3 of a concentration region C1, C2, C3 is, the larger the catchment region of the respective segment 20-1, 20-2, 20-3 is and the more marked cells 26 from the respective concentration region C1, C2, C3 are directed over and counted by the sensor 18. Accordingly, from the time t3, when the enriched marked cells 26 from the concentration region C2 flow over the sensor 18, the sensor 18 measures more count events than previously. At the time t4, the strongly enriched fraction of the concentration region C1 is measured.

The volumes of the individual concentration regions C1, C2, C3, C4 and the width b of the corresponding segment 20-1, 20-2, 20-3 are known or may easily be measured. The cell sample concentration may be calculated for each concentration region C1, C2, C3, C4 with the aid of the established cell number for each concentration region C1, C2, C3, C4. Ideally, the values should be the same.

Additionally, a time t1, t2, t3 or t4 of the counting process may be established. Consequently, a calibrated device may be used to establish the concentration region C1, C2, C3, C4 from which the cells 26 are currently being counted. The concentration region C1, C2, C3, C4 may be determined in a calibrated device 10 on the basis of the measured time. An established count frequency f (i.e., the distance between the marked cells 26), depends on the magnetic force, the flow speed and the cell concentration. The count frequency f decreases with decreasing concentration of the sample. By way of example, frequencies “f=1”, “f=2”, “f=3” and “f=4” are established in FIG. 6. The count frequency f may likewise be used for establishing the concentration of the cell sample 30. Additionally or alternatively, it is possible to establish a duration t*, in which a predetermined count frequency f is present. The duration t* depends on the selected length a of the respective segments 20-1, 20-2, 20-3 of the deflection device 20.

By using reference samples with a known cell concentration, a person skilled in the art is able to calibrate the count frequency f or the times t1, t2, t3, t4 for all concentration regions C1, C2, C3, C4 to the flow speed and the magnetic force.

In a possible calibration process, it is possible, for example, to set a range of flow speeds. Then, the time period t in which a defined volume and therefore a specific concentration region C1, C2, C3, C4 of the marked cells 26 was carried out is established when measuring the cell sample 30.

Depending on the cell concentration, the frequency f sets in at the corresponding time t1, t2, t3 or t4 in each concentration region C1, C2, C3, C4. The calibration is preferably performed for each concentration region. Ideally, each concentration region for current sensors is calibrated to 1000 count events in order to obtain stable statistics.

When measuring the desired cell sample 30 with an unknown concentration, use may then preferably be made of the measurement of that concentration region C1, C2, C3, C4, in which the count frequency lies near 1000, preferably additionally below 1000.

In summary, an embodiment may also be described as follows:

In order to quantify specific particles, in particular cells, within a complex sample 30, these have to be marked first with a marking that is specific to the cells. The required markers include, for example, a superparamagnetic material and are modified by antibodies on the surface thereof. This marking may specifically bind to the target particles. This act of marking is to be carried out, for example, within a complex sample 30 and requires no subsequent purification of the sample. The marking described here is brought about, for example, using superparamagnetic markers.

By stirring processes, the marking may mark all sought-after cells (i.e., 100% of the sought-after cells), within a sample 30. However, the marking may also be brought about partially (e.g. 1%). In the process, the marked antibodies are, for example, initially introduced into the channel 14 and enriched at one side of the channel 14 by an external magnetic field. When the sample 30 with particles or cells is subsequently introduced into the channel 14, only those cells or particles that are currently situated at the side of the channel 14 come into contact with the markers. In the case of a stochastic distribution, a specific portion (e.g. 1%) may be marked by a suitable design of the channel 14. All other particles do not come into contact with the marking.

Focusing by Magnetic and/or Mechanical Forces Under Laminar Flow Conditions:

Under the precondition of a superparamagnetic marking of the desired particles, the enrichment is brought about, for example, by a combination of magnetic forces (shown here in the z-direction: see FIG. 2A) and mechanical focusing by suitable structures (y-direction: see FIGS. 2A and 2B). A permanent magnet 16 or an electromagnet 16 (FIG. 2) may be positioned at the side at which an enrichment of the particles is desired. If a cell suspension 30 is introduced into the channel 14 (FIG. 4A), the marked cells or particles 26 are distributed stochastically. Magnetic forces move the marked cells or particles 26 to one side of the channel 14 (FIG. 4B), in this case in the z-direction.

As a result of the suitable design of the deflection device 20 (length a and width b) (FIG. 2B) at one of the inner sides of the utilized channel 14, it is possible to enrich or focus the cells or particles 26 in the y-direction at any position of the channel ball (here: at y=½).

The deflection device 20 may be, for example, barriers made of, for example, a photoresist that mechanically guide the cells or particles 26, or else the deflection device 20 may be made of ferromagnetic fishbone structures, which magnetically enrich and focus the marked cells or particles 26. A combination of both processes is also possible. FIG. 5 shows an example of such an enrichment path. FIG. 5A shows, in an exemplary manner, the stochastic distribution of marked cells 26 over the deflection device 20 immediately after introducing the sample 30. FIG. 3B shows the principle of focusing the marked cells 26 in the middle of the channel 14 at the times t1-t3.

Resolving a Plurality of Powers of 10:

If the channel height h (FIG. 2A) is varied as desired over the different concentration regions C1, C2, C3, C4 (FIG. 2B), for example, the cell number in this region of the channel 14 is likewise varied. As a result, it is possible to set a defined minimum number of cells per concentration region C1, C2, C3, C4. This procedure allows a statistically meaningful cell number to be set.

In the case of a partial marking, the height h is preferably the same in all concentration regions C1, C2, C3, C4. Only the layer that is situated on the side to which the marked antibodies were also pulled, for example, by an external magnetic field, is marked and only enriched to a different extent by the concentration regions C1, C2, C3, C4. As a result of the freely selectable width b and length a of the deflection device 20 (FIG. 2B), it is possible to enrich a defined fraction of the sample 30 (e.g. 50% of the channel width at C3) in the center of the channel 14.

Establishing the Cell or Particle Concentration:

It is possible to establish the concentration of the particles within a sample 30 by determining two parameters. The first parameter is the count frequency f in the x-direction of the focused cells 26 or, expressed differently, the distance between the particles 30. The count frequency f depends on the magnetic force, the flow speed and the cell or particle concentration. It is therefore possible to calibrate the count frequency f for all concentration regions C1, C2, C3, C4 to the flow speed and the magnetic force. The second parameter is the time t at which a specific frequency f sets in. Additionally, the time duration t*, in which a specific frequency f is counted, provides information about the present concentration region C1, C2, C3, C4. The time t and the duration t* are dependent on the selected length a of the respective deflection means 20. In general, the count frequency f is lower, the lower the concentration of the sample 30 is.

A calibrated system is a precondition. That is to say a range of flow speeds {v1; vn} that enable quantification of the focused, isolated cells 26 in a subsequent act are set. Thus, it is possible to establish the time period in which a defined volume, and hence a specific concentration region C1, C2, C3, C4 of the cell sample 30, was performed. So that a subsequent count system may count, the application may be calibrated to a specific count frequency f. Depending on the cell or particle concentration, this frequency f sets in at the corresponding time t1, t2, t3 or t4 in the concentration regions C1, C2, C3 or C4. It is advantageous if the calibration is performed for each concentration region C1, C2, C3, C4. Thus, an ideal calibration renders it possible that a subsequent quantification process resolves the concentration regions C1-C4. Ideally, each calibration of the concentration region C1, C2, C3 or C4 consists of up to 1000 counted cells in order to obtain stable statistics.

Determining the Concentration:

The concentration region C1, C2, C3, C4 of the sample may be determined by way of a specific count frequency f and a time range t. From the known geometry and the liquid volume in this concentration region C1, C2, C3, C4 resulting therefrom, it is possible to quantify cells or particles per volume. The counted cells or particles in this time window render it possible to deduce a concentration of the cells or particles in the whole sample 30.

Procedures During the Evaluation:

Firstly, it is possible to establish after what time t a count frequency f has been reached. As a result of this, it is possible to establish the concentration region C1, C2, C3, C4, which is calibrated to 1000 counted cells 26 or particles per measurement. It is likewise possible to establish the count value for this concentration region C1, C2, C3, C4.

A further embodiment of the cytometric concentration measurement may be explained on the basis of FIG. 6: A sample 30 that was marked by superparamagnetic markers and that has an unknown concentration is present. As a result of the described geometric parameters (the flow speed and/or the strength of the magnetic field), it is possible to set a desired frequency f of, for example, the marked cells 26 for the purposes of ideal quantification. FIG. 6 shows an example in which the system is calibrated to f=4. It is possible to see that the desired frequency f sets in at the time t=3. Therefore, this is a sample 30 with the concentration region C2. If the frequency f increases beyond 4, the count is completed because the cells 26 in the enrichment region C1 at the time t=4 are already being counted.

Some Measurement Examples, in which the Concentration Regions are Calibrated to 1000 Cells:

When calibrating the concentration region C1, which for example has a volume of 10 microliters, use is made of, for example, a reference sample with a concentration of 10² cells/microliter. By way of example, 300 cells are counted from the unknown sample 30; consequently, the unknown sample has a concentration of 30 cells/microliter.

A calibration of the concentration region C2 (1 microliter, concentration of the reference sample: 10³ cells/microliter) is followed by, for example, a measurement of, for example, 400 cells. The unknown sample has a concentration of 400 cells/microliter.

In the case of the calibration of the concentration region C3 (e.g. 0.1 microliter), the reference sample has, for example, a concentration of 10⁴ cells/microliter. 200 cells are counted in the unknown sample 30; the sought-after concentration is 2000 cells/microliter.

After calibrating the concentration region C4 (e.g., 0.01 microliter, concentration of the reference sample: 10⁵ cells/microliter), for example 800 cells are counted in a sample 30; consequently, the unknown sample has a concentration of 80 000 cells/microliter.

1. A device for a flow cytometric measurement, the device comprising:
a chamber;
a channel, wherein the channel comprises a magnetic sensor and is disposed downstream of the chamber and present with the chamber in a closed system, wherein an axis of the channel extends along a flow direction of the channel;
a magnet; and
a deflection device arranged at a predetermined side of the channel and wherein the deflection device is divided into at least one segment and each segment is arranged in a concentration region of the channel, in which the deflection device is configured to guide cells toward the axis.

2. The device as claimed in claim 1, wherein the magnet is arranged at the predetermined side of the chamber.

3. The device as claimed in claim 1, wherein the at least one segment of the deflection device comprises guide elements arranged at an angle to the axis for guiding cells, which guide elements together or on their own form at least one funnel shape tapering in the flow direction.

4. The device as claimed in claim 3, wherein
at least one of the guide elements forms a V-shape tapering in the flow direction and wholly or partly comprises a ferromagnetic material, and/or
at least two of the guide elements are offset on the axis in the flow direction and thereby form a mechanical guide toward the axis.

5. The device as claimed in claim 3, wherein a magnetic web is arranged along the axis of the channel.

6. The device as claimed in claim 1, wherein there are at least two concentration regions including the concentration region, the concentration having a different configuration than another concentration region.

7. The device as claimed in claim 6, wherein the at least two concentration regions at a predetermined level of the channel differ between the predetermined side and a side opposite to the predetermined side.

8. The device as claimed in claim 6, wherein a channel width is constant and the at least two concentration regions differ by a predetermined width of the deflection device.

9. The device as claimed in claim 6, wherein each concentration region is characterized by a length of the respective segment of the deflection device along the axis of the channel.

10. A method for enriching cells of a cell type to be detected in a cell sample for flow cytometry, the method comprising:
providing magnetically marked cells of the cell type to be detected; and
enriching the marked cells in a channel of a device, wherein the marked cells are pulled onto a predetermined side of the channel by a magnet and at least some of the cells are guided by a deflection device to an axis which extends along a flow direction of the channel.

11. The method as claimed in claim 10, wherein providing the magnetically marked cells comprises marking the cells and wherein the marking comprises:
providing at least one antibody specific to the cell type to be detected, which antibody is connected with at least one magnetic marker, in the channel, and
generating a magnetic field in the channel using the magnet arranged at the predetermined side of the channel, and
wherein enriching comprises introducing a cell sample including the cells into the channel, bringing the at least one specific antibody into contact with that portion of the cell sample the flows past the predetermined side of the channel in a laminar manner, and thereby marking the cells contained in this portion.

12. The method as claimed in claim 10, wherein cytometry is performed on the axis downstream of the deflection device by a magnetic sensor.

13. The method as claimed in claim 12, wherein the concentration of the cell sample is determined by cytometry downstream of the deflection device, said determination comprising:
counting the enriched cells from at least one of multiple concentration regions, which cells are flowing past on the axis, by the magnetic sensor,
establishing the concentration of the cell sample for at least the one of the concentration regions from the counted value of the concentration region established when counting the enriched cells, the volume of the concentration region and the width of the deflection device.

14. The method as claimed in claim 13, wherein the one concentration region in which the cells are currently being counted is established on a basis of an established time of the counting by the magnetic sensor.

15. The device as claimed in claim 4, wherein a magnetic web is arranged along the axis of the channel.

16. The device as claimed in claim 15, wherein the channel includes at least two concentration regions including the concentration region, and wherein the at least two concentration regions have different configurations.

17. The device as claimed in claim 16, wherein the at least two concentration regions at a predetermined level of the channel differ between the predetermined side and a side opposite to the predetermined side.

18. The device as claimed in claim 17, wherein a channel width is constant and the at least two concentration regions differ by a predetermined width of the deflection device.

19. The device as claimed in claim 6, wherein each concentration region is characterized by a length of the respective segment of the deflection device along the axis of the channel.
